(19) United States
(12) Patent Application Publication
(10) Pub. No.: US 2005/0215837 A1
(43) Pub. Date: Sep. 29, 2005

(54) The utilization of zirconium and zirconium based alloys for the containment of halogen containing environments used in the production of olefins, alcohols, ethers, and olefin oxides from alkanes

(75) Inventors: Ronald Anthony Hoffpauir, Humble, TX (US); Lizbeth Olivia Cisneros Trevino, Stafford, TX (US)

Correspondence Address: SHELL OIL COMPANY, P O BOX 2463, HOUSTON, TX, 772522463, US

(73) Assignee: Shell Oil Company

(21) Appl. No.: 11/087,109

(22) Filed: Mar. 23, 2005

Related U.S. Application Data
(60) Provisional application No. 60/555,476, filed on Mar. 23, 2004.

(51) Int. Cl. C07C 27/16

(52) U.S. Cl. 568/910

(57) ABSTRACT

This invention relates to a process for the production of olefins, alcohols, ethers, and olefin oxides from alkanes in a halogen, preferably bromine or chlorine, system, wherein there are halogenation (reaction of halogen with the alkanes), oxidation (reaction of alkyl halide with a metal oxide), neutralization (reaction of hydrogen halide and metal oxide), and regeneration (reaction of metal halide with air, oxygen, or other oxygen gas containing mixtures) reactions which take place in the process and that these reactions, at least, are carried out in reactors made from metallurgy including zirconium and/or zirconium-based alloys that contain varying amounts of alloying elements such as tin, niobium, chromium, iron, oxygen, and nickel. Preferably, this same metallurgy is used in the fabrication of separation and purification equipment for the process.

### REFERENCE TO PRIOR APPLICATIONS

This application claims the benefit of U.S. Provisional application Ser. No. 60/555,476, filed Mar. 23, 2004.

### FIELD OF THE INVENTION

This invention relates to a process for manufacturing olefins, alcohols, ethers, and olefin oxides from alkanes by mixing an alkane and halide in the reactor to form alkyl halide and hydrogen halide wherein the alkyl halide is contacted with a metal oxide to form an olefin, alcohol, ether, or olefin oxide and metal halide. More particularly, this invention relates to a choice of materials for the reactors in which this process is carried out.

### BACKGROUND OF THE INVENTION

The engineering considerations regarding the industrial handling of halogen or halogen-containing mixtures are not trivial. Material of construction identification is critical for the commercial success of a new process chemistry involving halogens. For example, in Materials Selection for the Chemical Process Industries by C. P. Dillon, published by McGraw-Hill Inc. in 1992, there is a chapter on the production of acetic acid wherein part of the process involves the carbonylation of methanol and carbon monoxide in the presence of an iodine-complex catalyst. At page 176, it is stated that zirconium 702 is one of the materials which could be used in the reactor and flash tank to cope with acetic acid and iodine compounds at 150° C.

U.S. Pat. Nos. 4,278,810 and 5,847,203 discuss the problems with bromine catalyzed reactions for the production of terephthalic acid. In column 1 of both patents, it is stated that expensive titanium and titanium alloys have been used as construction materials in such plants to accommodate the corrosivity of the bromine systems. Both patents relate to process changes which allow the use of stainless steel instead of titanium.

U.S. Pat. No. 4,330,676 describes another such process and at column 4 states that when the catalyst contains a bromide, a material must be used for withstanding the resulting highly corrosive reaction mixture and titanium is given as the example.

According to publicly available information (e.g., U.S. Pat. No. 6,403,840 B1, U.S. Pat. No. 6,462,243 B1, U.S. Pat. No. 6,465,696 B1, U.S. Pat. No. 6,465,699 B1, U.S. Pat. No. 6,472,572 B1, U.S. Pat. No. 6,486,368 B1, and U.S. Pat. No. 6,525,230 B2, etc., which are herein incorporated by reference), a process exists which consists of mixing an alkane and a halide in a reactor to form alkyl halide and hydrogen halide. The isolated alkyl halide or the alkyl halide and hydrogen halide mixture react with a metal oxide to produce the products (alcohols, ethers, olefins, or olefin oxide) and metal halide. The metal halide is oxidized with oxygen or air to form the original metal oxide and halide, both of which are recycled.

The hydrogen halide and/or the alkyl halide, when contacted with a metal oxide, will likely produce byproducts/products such as water and hydrogen halide. The combination of these constituents reacted at temperatures above 100° C. results in an environment that is highly corrosive to most of the commonly used metals such as carbon steel, stainless steels, and duplex stainless steels. This type of environment is especially corrosive in areas in which a liquid aqueous phase may exist. Some of the more exotic metals that been proposed for this type of environment (See U.S. Pat. No. 5,847,203, U.S. Pat. No. 4,330,676, and U.S. Pat. No. 4,278,810) are titanium and Hastelloy C. However, recently generated test data presented in Example 1 below indicate that Hastelloy C, or more generically, the nickel-chrome-molybdenum alloy family, affords very little resistance to corrosion under conditions which simulated the corrosive conditions that are anticipated in this process environment.

According to the documents discussed above, titanium has been used to overcome the corrosivity of bromine reaction mixtures. Titanium is a reactive metal and it relies heavily on the integrity of a protective oxide layer to prevent corrosion damage. Within the process environment in the present process, there is an inherent presence of nascent, or unassociated, hydrogen atoms. Nascent hydrogen is known to penetrate the protective oxide layer and migrate into the matrix of a base metal. If enough hydrogen penetrates into the base metal, internal metal hydrides may form and these are often detrimental to the mechanical properties, as well as to the metal's ability to resist corrosion. This damage mechanism is commonly referred to as hydride embrittlement.

Historically, hydride embrittlement has been recognized as a problem in many titanium applications. However, the likelihood of hydride embrittlement of titanium is difficult to precisely quantify. Controlled laboratory testing of this phenomenon is very difficult since the onset of hydride formation may take one year or longer.

### SUMMARY OF THE INVENTION

In one embodiment, the present invention relates to a process for the production of olefins, alcohols, ethers, and olefin oxides from alkanes (paraffins) in a halogen, preferably bromine or chlorine, system, wherein there are halogenation (reaction of halogen with the alkanes), oxidation (reaction of alkyl halide with a metal oxide), neutralization (reaction of hydrogen halide and metal oxide), and regeneration (reaction of metal halide with air, oxygen, or other oxygen gas containing mixtures) reactions which take place in the process and that these reactions, at least, are carried out in reactors made from metallurgy including zirconium and/or zirconium-based alloys that contain varying amounts of alloying elements such as tin, niobium, chromium, iron, oxygen, and nickel. Preferably, this same metallurgy is used in the fabrication of separation and purification equipment for the process.

Another embodiment of the present invention describes a process for the production of alcohols, olefins, ethers, and olefin oxides from alkanes which comprises the steps of:
a) halogenating an alkane to produce a mixture of alkyl halides (mono-haloalkanes and possibly multi-haloalkanes), unreacted alkanes, and hydrogen halide, preferably wherein the halogenation step may be carried out thermally and/or catalytically;
b) oxidizing the alkyl halide (or a subset of the alkyl halides such as primary mono-haloalkanes), optionally together with the hydrogen halide, with a metal oxide to produce the reaction products and metal halide, wherein the hydrogen halide is optionally separated;
c) if the hydrogen halide is separated in step b), neutralizing it with a metal oxide to produce a metal halide; and
d) regenerating the metal halide(s) from the oxidation and neutralization steps b) and c) to metal oxide and halogen using oxygen, air, or a gas mixture containing oxygen gas (these mixtures may include blends of oxygen with nitrogen, argon, or helium) such that the halogen and metal oxide may be recycled for use in the process; wherein these steps are carried out in equipment which is made from metallurgy which includes zirconium and/or zirconium based alloys. The zirconium-based alloys may contain varying amounts, preferably from 0.01 to 3% by weight of the total weight of the alloy, of alloying elements such as tin, niobium, chromium, iron, oxygen, and nickel. Preferably, other steps of the process where halogen and water may coexist are also carried out in equipment made with such metallurgy. Preferably, this same metallurgy is used in the fabrication of separation and purification equipment for the process.

In another embodiment, the invention is a process for the production of alpha olefins. The process converts branched or n-alkanes to branched or linear alpha olefins (AO) of the same carbon number. The halogenation, oxidation, neutralization, and regeneration reactions, at least, are carried out in reactors made from the metallurgy described in the preceding embodiment.

In a further embodiment, the invention is a process for the conversion of linear, branched or a mixture of linear and branched alkanes into alpha olefins. It comprises the steps of:
a) halogenating linear alkanes, branched alkanes, or a mixture of linear and branched alkane(s) to produce a mixture of primary mono-haloalkanes (i.e., alkanes with one halogen attached in the primary position), internal mono-haloalkanes (i.e., alkanes with one halogen attached somewhere other than the primary position), unreacted alkanes, hydrogen halide, and possibly multi-haloalkanes (i.e., alkanes with 2 or more halogens attached), preferably wherein the halogenation may be carried out thermally or catalytically;
b) separating the primary mono-haloalkanes from the mixture of step a) by distillation or other appropriate separation step(s);
c) separating the hydrogen halide produced in the halogenation step a) and neutralizing it with a metal oxide or mixture of metal oxides to produce a partially halogenated metal oxide and/or metal halide or mixture of partially halogenated metal oxides and/or metal halides which are then converted for recycle to halogen and metal oxide (or mixture of metal oxides) using air, oxygen, or gas mixtures containing oxygen gas (these mixtures may include blends of oxygen with nitrogen, argon, or helium);
d) oxidizing the separated primary mono-haloalkane with a metal oxide or combination of metal oxides to convert the aforesaid primary mono-haloalkane to a mixture of products that contains alpha olefins, unconverted primary mono-haloalkanes, and possibly other reaction products (such as internal olefins, primary alcohols and internal alcohols), and a partially halogenated metal oxide and/or metal halide or a mixture of partially halogenated metal oxides and/or metal halides;
e) separating and regenerating the partially halogenated metal oxide and/or metal halide or mixture of partially halogenated metal oxides and/or metal halides from step d) to a metal oxide or mixture of metal oxides and molecular halogen (such as Cl₂) by reaction with air, oxygen, or gas mixtures containing oxygen gas (these mixtures may include blends of oxygen with nitrogen, argon, or helium) wherein the halogen produced and/or the metal oxide may be recycled; and
f) removing the unreacted primary mono-haloalkane from the reaction mixture and then purifying the alpha olefin; wherein steps a), c), d), and e) are carried out in equipment which is made from metallurgy which includes zirconium and/or zirconium based alloys. The zirconium-based alloys may contain varying amounts, preferably from 0.01 to 3% by weight of the total weight of the alloy, of alloying elements such as tin, niobium, chromium, iron, oxygen, and nickel. Preferably, other steps of the process where halogen and water may coexist are also carried out in equipment made with such metallurgy. Preferably, this same metallurgy is used in the fabrication of separation and purification equipment for the process.

In another embodiment, there is described a process to convert alkanes to primary alcohols of the same carbon number wherein the halogenation, oxidation, and regeneration, at least, are carried out in reactors made from metallurgy including zirconium and zirconium-based alloys that contain varying amounts of alloying elements such as tin, niobium, chromium, iron, oxygen, and nickel. Preferably, this same metallurgy is used in the fabrication of separation and purification equipment for the process.

This embodiment describes a process for the production of primary alcohols from alkanes which comprises the steps of:
a) halogenating a linear or branched (or mixture of linear and branched) alkane to produce a mixture of primary mono-haloalkanes (i.e., alkanes with one halogen attached in the primary position), internal mono-haloalkanes (i.e., alkanes with one halogen attached somewhere other than the primary position), unreacted alkanes, hydrogen halide, and possibly multi-haloalkanes (i.e., alkanes with 2 or more halogens attached), preferably wherein the halogenation may be carried out thermally or catalytically;
b) separating the primary mono-haloalkanes from the mixture of step a) by distillation or other appropriate separation step(s);
c) oxidizing the separated primary mono-haloalkane with a metal oxide or combination of metal oxides and water (and possible hydrogen halide) to convert the aforesaid primary mono-haloalkane to a mixture of products that contains primary alcohols, unconverted primary mono-haloalkanes, and possibly other reaction products (such as internal alcohols and/or olefins), and a partially halogenated metal oxide and/or metal halide or a mixture of partially halogenated metal oxides and/or metal halides;
d) separating and regenerating the partially halogenated metal oxide and/or metal halide or a mixture of partially halogenated metal oxides and/or metal halides to a metal oxide or mixture of metal oxides and molecular halogen (such as Cl₂) by reaction with air, oxygen or gas mixtures containing oxygen gas (these mixtures may include blends of oxygen with nitrogen, argon, or helium), wherein the halogen produced and/or the metal oxide may be recycled; and
e) removing the unreacted primary mono-haloalkane from the reaction mixture and then purifying the primary alcohol; wherein steps a), c) and d) are carried out in equipment which is made from metallurgy which includes zirconium and/or zirconium based alloys. The zirconium-based alloys may contain varying amounts, preferably from 0.01 to 3% by weight of the total weight of the alloy, of alloying elements such as tin, niobium, chromium, iron, oxygen, and nickel. Preferably, other steps of the process where halogen and water may coexist are also carried out in equipment made with such metallurgy. Preferably, this same metallurgy is used in the fabrication of separation and purification equipment for the process.

### DETAILED DESCRIPTION OF THE INVENTION

The process of the present invention is applicable to the production of olefins, alcohols, ethers, and olefin oxides from alkanes of almost any carbon number. The product carbon numbers of primary interest are C₁ to C₂₀ and the product carbon numbers of particular interest are C₈ to C₁₈.

Alkanes are converted via halogenation to a mixture of primary mono-haloalkanes, internal mono-haloalkanes, unreacted alkanes, hydrogen halide, and possibly multi-haloalkanes. Halogenation may preferably be carried out thermally or catalytically (for example in a conventional reactor, in a catalytic distillation (CD) column, etc.), and with or without the use of a support intended to promote shape selectivity. For the production of primary alcohols and alpha olefins, halogenation processes that preferentially produce primary mono-haloalkanes (e.g., catalytic halogenation at lower temperatures, thermal halogenation at higher temperatures, etc.) are preferred. Preferred halogens are chlorine, bromine, and iodine. For the production of primary alcohols and alpha olefins, chlorine is preferred. For other olefins, alcohols, ethers, and olefin oxides, bromine may be preferred.

Thermal halogenation is carried out by introducing the halogen and the alkane to a reactor. The reaction temperature may be from 100° C. to 400° C. As stated above, catalytic halogenation may be carried out at lower temperature, such as from 25° C. to 400° C. Catalysts which may be used include compounds of Ti, Zr, Hf, V, Nb, Ta, Cr, Mo, W, Mn, Re, Fe, Ru, Co, Rh, Ir, Ni, Pd, Pt, Cu, Ag, Au, Zn, Cd, B Al, Ga, In, Tl, Si, Ge, Sn, Pb, P, Sb, Bi, S, Cl, Br, F, Sc, Y, Mg, Ca, Sr, Ba, Na, Li, K, 0, La, Ce, Pr, Nd, Sm, Eu, Gd, Tb, Er, Yb, Lu and Cs or mixtures thereof.

For the case of primary alcohols and alpha olefins, the mixture of primary mono-haloalkanes, other mono- and multi-haloalkanes, unreacted alkanes, and hydrogen halide is transferred to a separation train that isolates the primary mono-haloalkanes from the mixture. To produce primary alcohols and/or alpha olefins, it is preferred to direct only primary mono-haloalkanes to the oxidation reactor. The separation train may include (1) a distillation or other appropriate separation step to recover hydrogen halide, (2) a distillation or other appropriate separation step (or multiple steps) to separate unreacted alkanes, multi-haloalkanes, and mono-haloalkanes, and (3) an additional separation step to separate primary mono-haloalkanes from internal mono-haloalkanes. The unreacted alkanes may be recycled to the primary halogenation reactor. The multi-haloalkanes may be recycled to the primary halogenation reactor or may be recycled to a disproportionation reactor to convert some of the multi-haloalkanes to mono-haloalkanes. If a disproportionation reactor is used, the resulting reaction mixture of multi-haloalkanes and mono-haloalkanes is then recycled to the separation train. The internal mono-haloalkanes may be recycled to the primary halogenation reactor or may be recycled to an isomerization reactor to convert some of the internal mono-haloalkanes to primary mono-haloalkanes. If an isomerization reactor is used, the resulting reaction mixture of internal alkyl halides and primary alkyl halides is then recycled to the separation train.

Suitable separation schemes include distillation, adsorption, melt crystallization, and others. For the primary and internal mono-haloalkanes separation, distillation and melt crystallization are particularly preferred. For some carbon chain lengths (C₆-C₁₀), distillation is preferred because of differences in boiling points (and as result, relative volatilities). For other carbon chain lengths (C₁₂-C₁₆), melt crystallization is preferred because of the substantial freezing point difference between primary and internal mono-haloalkanes.

The hydrogen-halide produced in the halogenation reactor may be separated and neutralized with a metal oxide to produce a metal halide. Engineering configurations to carry out this hydrogen halide neutralization process include a single reactor, parallel reactors, and two reactors (one to trap hydrogen halide and one to regenerate metal-halide), among others. Using air, oxygen, or other oxygen gas containing mixtures (these mixtures may include blends of oxygen with nitrogen, argon, or helium), this metal halide is converted (regenerated) to halogen and the original metal oxide both of which are preferably recycled.

Another option for using the hydrogen halide is to send it to a metathesis reactor (also called an oxidation reactor), where alkyl-halides are reacted with metal oxide as explained below. Metal oxides which may be used in this step and in the other metathesis reaction below, include oxides of the following metals: Ti, Zr, Hf, V, Nb, Ta, Cr, Mo, W, Mn, Re, Fe, Ru, Co, Rh, Ir, Ni, Pd, Pt, Cu, Ag, Au, Zn, Cd, B, Al, Ga, In, Tl, Si, Ge, Sn, Pb, P, Sb, Bi, S, Cl, Br, F, Sc, Y, Mg, Ca, Sr, Ba, Na, Li, K, La, Ce, Pr, Nd, Sm, Eu, Gd, Tb, Er, Yb, Lu, and Cs or mixtures thereof.

The alkyl-halide (primary mono-haloalkane for the production of alpha-olefins and/or primary alcohols) that is isolated in the separation train alone or produced in the halogenation reactor along with the hydrogen halide is sent into a metathesis reactor with a selected metal oxide or a combination of metal oxides to convert the alkyl-halide to a mixture of products. The product distribution of olefins, alcohols, ethers, and/or olefin oxides depends on the metal oxide used in the metathesis reaction.

Water may be fed to the reactor to aid in the formation of alcohols by providing an extra source of hydrogen and/or oxygen. The reaction conditions such as residence time, temperature, reaction phase (solid-gas, solid-liquid, etc.), and addition of water or hydrogen halide are selected to maximize the desired product production. The same metal oxide or combination of metal oxides may be able to produce preferentially different products (such as alcohols instead of olefins, ethers or olefin oxide) depending on the reaction conditions. For example, longer residence times, higher temperatures, and solid-liquid phase reaction tend to preferentially produce alcohols over olefins. The addition of water to the metathesis reaction may be crucial for the production of alcohols.

The metal oxide or metal oxides used in the metathesis reactor may or may not be different from the one(s) used in the neutralization of the hydrogen halide. The metal oxide is partially (or totally) converted to a metal halide. A purification train is used to isolate the product. Suitable purification schemes include distillation, adsorption, melt crystallization, and others. The unconverted alkyl-halides are recycled to the metathesis reactor.

The metal halide is regenerated to metal oxide and halide by using air, oxygen, or a mixture oxygen gas containing gas (these mixtures may include blends of oxygen with nitrogen, argon, or helium). The liberated halogen is preferably recycled to the halogenation reactor. The regeneration of metal halide to metal oxide and halide may be accomplished with various reactor configurations including a separate regeneration reactor, in situ with a combined regeneration/metathesis reactor where the air/oxygen flow and primary alkane feed flow are alternated (for example, as described in U.S. Pat. No. 6,525,230, which is herein incorporated by reference), in situ regeneration with a multiple metathesis reactor configuration in a fixed bed mode, etc. Irrespective of reactor design, type of metal oxide, or halogen, zirconium metallurgy is suited for the regeneration reactor.

The final product (olefins, alcohols, ethers or olefins) is purified in a separation train.

The present invention offers a family of suitable metals for the containment of the type of hot wet halogen containing environments (especially chlorine and bromine) that exist in parts of this process of reacting alkanes to form olefins, alcohols, ethers and/or olefin oxides. This invention identifies this metallurgy as suitable for use in the fabrication of separation equipment that could be utilized in the purification of above-mentioned products. The specific metallurgy identified includes zirconium and zirconium based alloys that contain varying amounts of alloying elements such as tin, niobium, chromium, iron, oxygen, and nickel.

Generally, the alloying elements described above are present in the zirconium in amounts ranging from 0.01 to 3 percent by weight of the total alloy. A partial list of these types of zirconium alloys includes zirconium 702 (aka UNS Grade R60702), zirconium 704 (aka UNS Grade R60704), zirconium 705 (aka UNS Grade R60705), zirconium 706 (aka UNS Grade R60706), zirconium 702-S, Zr-2.5 Nb (aka UNS Grade R60901), Zircaloy-2 (aka UNS Grade R60802), and Zircaloy-4 (aka UNS Grade R60804).

The chemical requirements of many of these zirconium based alloys are provided in the American Standards for Testing and Materials (ASTM) standard B 551. The chemical composition requirements for some of these materials expressed in weight percent (wt %), as provided in ASTM B-551 are as follows: zirconium 702—99.2 minimum wt % Zr+Hf, 0.05 maximum wt % C, 0.2 maximum wt % F+Cr, 0.005 maximum wt % H, 4.5 maximum wt % Hf, 0.025 maximum wt % N, and 0.16 maximum wt % oxygen; zirconium 704—97.5 minimum wt % Zr+Hf, 0.05 maximum wt % C, 0.2-0.4 wt % Fe⁺ Cr, 0.005 maximum wt % H, 4.5 maximum wt % Hf, 0.025 maximum wt % N, 0.18 maximum wt % oxygen, and 1.0-2.0 wt % Sn; zirconium 705—95.5 minimum wt % Zr+Hf, 0.05 maximum wt % C, 0.2 maximum wt % Fe+Cr, 0.005 maximum wt % H, 4.5 maximum wt % Hf, 0.025 maximum wt % N, 2.0-3.0 wt % Nb, and 0.18 maximum wt % oxygen; and zirconium 706—95.5 wt % Zr+Hf, 0.05 maximum wt % C, 0.2 maximum wt % Fe⁺ Cr, 0.005 maximum wt % H, 4.5 maximum wt % Hf, 0.025 maximum wt % N, 2.0-3.0 wt % Nb, and 0.16 maximum wt % oxygen.

Zirconium 702-S is a designator assigned to a recently developed variation on zirconium 702 that sets a more rigorous requirement on the amount of Sn that is allowed in the requirements for zirconium 702. The maximum content of Sn that is allowed in zirconium 702-S is 0.25 wt % Sn. Otherwise, the chemical requirements for zirconium 702-S are identical to zirconium 702. The chemical requirements for this new metal were obtained from a zirconium manufacturer's website—www.wahchang.com.

Zircaloy-2 (aka UNS Grade R60802) and Zircaloy-4 (aka UNS Grade R60802) are both common zirconium-tin (Sn) alloys. The American Society of Metals (ASM) Handbook, volume 2, provides a typical composition for these zirconium-tin alloys as follows: Zircaloy-2—1.4 wt % Sn, 0.1 wt % Fe, 0.1 wt % Cr; 0.05 wt % Ni; 0.12 wt % 0, and the balance Zr; and Zircaloy-4—1.4 wt % Sn, 0.2 wt % Fe, 0.1 wt % Cr, 0.12 wt % 0, and the balance Zr.

Zr-2.5 Nb (aka UNS Grade R60901) is a common zirconium-niobium (Nb) alloy. The American Society of Metals (ASM) Handbook, volume 2, provides a typical composition for this zirconium-niobium alloy as follows: Zr-2.5Nb—2.6 wt % Nb, 0.14 wt % 0, and the balance Zr.

The hydrogen halide and/or the alkyl halide, when contacted with a metal oxide, may produce byproducts/products such as water and hydrogen halide. The hot process environment required will contain water as well as the halogen(s), preferably bromine or chlorine. The combination of these constituents reacted at temperatures above 100° C. results in an environment that is highly corrosive to most of the commonly used metals such as carbon steel, stainless steels, and duplex stainless steels. The environment of this process is especially corrosive in areas in which a liquid aqueous phase may exist. Some of the more exotic metals that been proposed for this type of environment (See U.S. Pat. No. 5,847,203, U.S. Pat. No. 4,330,676, and U.S. Pat. No. 4,278,810) are titanium and Hastelloy C. However, recently generated test data presented in Example 1 indicate that Hastelloy C, or more generically the nickel-chrome-molybdenum alloy family, affords very little resistance to corrosion under conditions which are similar to the corrosive conditions in the environment of this process. The results from these same tests indicate that zirconium based metals offer adequate corrosion resistance and are suitable materials of construction for this processes.

A comparison of the chemical properties and industrial experience between titanium and zirconium further supports the position that zirconium and its alloys are more suitable alternatives for this process environment. Both of these metals are classified as reactive metals and they rely heavily on the integrity of a protective oxide layer to prevent corrosion damage. Within this process environment there is an inherent presence of nascent, or unassociated, hydrogen atoms. Nascent hydrogen is known to penetrate the protective oxide layer and migrate into the matrix of the base metal. The ability of the zirconium to facilitate the transport of hydrogen harmlessly through the metal matrix is better than that of titanium. The solubility of hydrogen in zirconium is much lower than that of titanium.

The degree of solubility of hydrogen in the base metals relates directly to the susceptibility of the base metals to form internal metal hydrides, which are often detrimental to the mechanical properties, as well as to the metal's ability to resist corrosion. This damage mechanism is commonly referred to as hydride embrittlement.

Historically hydride embrittlement has been a recognized problem in many titanium applications. However, the likelihood of hydride embrittlement of titanium is difficult to precisely quantify. Controlled laboratory testing of this phenomenon is very difficult since the onset of hydride formation may take one year or longer. Consequently, much of the data that relates to hydride embrittlement of titanium is anecdotally based on field experiences. However a study of relevant case histories suggests to us that titanium metal that is exposed to dry, or slightly wet, highly acidic environments is prone to this form of damage. Based on this criterion, we consider hydride embrittlement of titanium to be a significant concern for the present process environment.

Thus, it appears that titanium should not be chosen as the metallurgy used in the process of the present invention because of the significant risk factor. Zirconium, with its ability to facilitate the transport of hydrogen harmlessly through the metal matrix and the lower solubility of hydrogen in zirconium, is a much better choice.

### EXAMPLES

Short term corrosion testing was performed in an attempt to find acceptable materials for this process environment. These corrosion tests were conducted in four cells containing water that was saturated with bromine. Each of the cells were constantly stirred and maintained at a high enough pressure to ensure the water remained in the liquid state. The tests were run at two temperatures, 150° C. and 188° C. These tests simulated water condensation at those high temperatures.

Oxygen may have a very dramatic effect on the corrosion rates of many metals. Since various areas of the proposed process will have varying contents of oxygen, one set of tests was initially purged of oxygen by displacement with nitrogen gas, while the second set allowed for the presence of oxygen contamination.

The tests were originally scheduled to run for 10 days. The thermocouples used to control the temperature of one of the test cells failed due to corrosion after only three days of operation. This forced the immediate shut down of this test cell. Upon inspection of the coupons that were retrieved from this cell it was determined that the integrity of the remaining test cells, which were constructed of Hastelloy C276, might have been compromised if the testing were to continue for the entire 10 day duration. Due to this concern the tests in the three remaining cells were subsequently terminated.

Although the test duration was abbreviated, the corrosion data reveals a significant advantage in the corrosion resistance of Zirconium 702 in comparison to the more commonly used nickel and chrome alloys. The data from these tests are provided in the table below.

It should be noted that although this testing targeted a hot bromine/water environment, similar trends in data are expected for the analogous chlorine based environment.

TABLE 1
Corrosion Testing in Hot Aqueous Bromine Environments

| | | Oxygen | | | | Inches of |
|---|---|---|---|---|---|---|
| | | Present | | Test | Corrosion Rate | Metal Loss |
| Test # | Temperature | (Yes/No) | Metal | Duration | (1 mpy = 0.001″ per year) | per Year |
| 1 | 188° C. | Yes | Type 304L SS | 125 hrs | 2372 mpy | 2.37 |
| 2 | 188° C. | Yes | Hastelloy B2 | 125 hrs | 443 mpy | 0.44 |
| 3 | 188° C. | Yes | Hastelloy C276 | 125 hrs | 86 mpy | 0.09 |
| 4 | 188° C. | Yes | Inconel 625 | 125 hrs | 187 mpy | 0.19 |
| 5 | 188° C. | Yes | Zirconium 702 | 125 hrs | 0.32 mpy | 0.0003 |
| 6 | 150° C. | Yes | Type 304L SS | 125 hrs | 1397 mpy | 1.40 |
| 7 | 150° C. | Yes | Hastelloy B2 | 125 hrs | 586 mpy | 0.59 |
| 8 | 150° C. | Yes | Hastelloy C276 | 125 hrs | 104 mpy | 0.10 |
| 9 | 150° C. | Yes | Inconel 625 | 125 hrs | 144 mpy | 0.14 |
| 10 | 150° C. | Yes | Zirconium 702 | 125 hrs | 0.36 mpy | 0.0004 |
| 11 | 188° C. | No | Type 304L SS | 101 hrs | 3126 mpy | 3.22 |
| 12 | 188° C. | No | Hastelloy B2 | 101 hrs | 493 mpy | 0.49 |
| 13 | 188° C. | No | Hastelloy C276 | 101 hrs | 150 mpy | 0.15 |
| 14 | 188° C. | No | Inconel 625 | 101 hrs | 331 mpy | 0.33 |
| 15 | 188° C. | No | Zirconium 702 | 101 hrs | 1.04 mpy | 0.001 |
| 16 | 150° C. | No | Type 304L SS | 101 hrs | 543 mpy | 0.54 |
| 17 | 150° C. | No | Hastelloy B2 | 101 hrs | 1160 mpy | 1.16 |
| 18 | 150° C. | No | Hastelloy C276 | 101 hrs | 38 mpy | 0.04 |
| 19 | 150° C. | No | Inconel 625 | 101 hrs | 81 mpy | 0.08 |
| 20 | 150° C. | No | Zirconium 702 | 101 hrs | 0.54 mpy | 0.0005 |

1. A process for the production of olefins, alcohols, ethers, and olefin oxides from alkanes in a halogen system, wherein there are halogenation (reaction of halogen with the alkanes), oxidation (reaction of alkyl halide with a metal oxide), neutralization (reaction of hydrogen halide and metal oxide), and regeneration (reaction of metal halide with air, oxygen, or other oxygen gas containing mixtures) reactions which take place in the process and that these reactions, at least, are carried out in reactors made from metallurgy including zirconium and/or zirconium-based alloys that contain varying amounts of alloying elements such as tin, niobium, chromium, iron, oxygen, and nickel.

2. The process of claim 1 wherein the zirconium-based alloys may contain varying amounts, preferably from 0.01 to 3% by weight of the total weight of the alloy, of alloying elements such as tin, niobium, chromium, iron, oxygen, and nickel.

3. A process for the production of alcohols, olefins, ethers, and olefin oxides from alkanes which comprises the steps of:
a) halogenating an alkane to produce a mixture of alkyl halides, unreacted alkanes, and hydrogen halide;
b) oxidizing the alkyl halide, optionally together with the hydrogen halide, with a metal oxide to produce the reaction products and metal halide, wherein the hydrogen halide is optionally separated;
c) if the hydrogen halide is separated in step b), neutralizing it with a metal oxide to produce a metal halide; and
d) regenerating the metal halide(s) from the oxidation and neutralization steps b) and c) to metal oxide and halogen using oxygen, air, or a gas mixture containing oxygen gas such that the halogen and metal oxide may be recycled for use in the process;
wherein these steps are carried out in equipment which is made from metallurgy which includes zirconium and/or zirconium based alloys.

4. The process of claim 3 wherein the gas mixture is selected from the group consisting of blends of oxygen with nitrogen and/or argon and/or helium.

5. The process of claim 3 wherein the zirconium-based alloys may contain varying amounts, preferably from 0.01 to 3% by weight of the total weight of the alloy, of alloying elements such as tin, niobium, chromium, iron, oxygen, and nickel.

6. The process of claim 3 wherein the metal halides produced in the oxidation and neutralization reactions are different and they are regenerated independently.

7. A process for the production of alpha olefins from branched or n-alkanes of the same carbon number in a halogen system, wherein there are halogenation (reaction of halogen with the alkanes), oxidation (reaction of alkyl halide with a metal oxide), neutralization (reaction of hydrogen halide and metal oxide), and regeneration (reaction of metal halide with air, oxygen, or other oxygen gas containing mixtures) reactions which take place in the process and that these reactions, at least, are carried out in reactors made from metallurgy including zirconium and/or zirconium-based alloys that contain varying amounts of alloying elements such as tin, niobium, chromium, iron, oxygen, and nickel.

8. The process of claim 7 wherein the zirconium-based alloys may contain varying amounts, preferably from 0.01 to 3% by weight of the total weight of the alloy, of alloying elements such as tin, niobium, chromium, iron, oxygen, and nickel.

9. A process for the production of alpha olefins from branched or n-alkanes of the same carbon number which comprises the steps of:
a) halogenating linear alkanes, branched alkanes, or a mixture of linear and branched alkanes to produce a mixture of primary mono-haloalkanes, internal mono-haloalkanes, unreacted alkanes, hydrogen halide, and possibly multi-haloalkanes;
b) separating the primary mono-haloalkanes from the mixture of step a) by distillation or other appropriate separation step(s);
c) separating the hydrogen halide produced in the halogenation step a) and neutralizing it with a metal oxide or mixture of metal oxides to produce a partially halogenated metal oxide and/or metal halide or mixture of partially halogenated metal oxides and/or metal halides which are then converted for recycle to halogen and metal oxide (or mixture of metal oxides) using air, oxygen, or gas mixtures containing oxygen gas;
d) oxidizing the separated primary mono-haloalkane with a metal oxide or combination of metal oxides to convert the aforesaid primary mono-haloalkane to a mixture of products that contains alpha olefins, unconverted primary mono-haloalkanes, and possibly other reaction products, and a partially halogenated metal oxide and/or metal halide or a mixture of partially halogenated metal oxides and/or metal halides;
e) separating and regenerating the partially halogenated metal oxide and/or metal halide or mixture of partially halogenated metal oxides and/or metal halides from step d) to a metal oxide or mixture of metal oxides and molecular halogen by reaction with air, oxygen, or gas mixtures containing oxygen gas wherein the halogen produced and/or the metal oxide may be recycled; and
f) removing the unreacted primary mono-haloalkane from the reaction mixture and then purifying the alpha olefin;
wherein steps a), c), d), and e) are carried out in equipment which is made from metallurgy which includes zirconium and/or zirconium based alloys.

10. The process of claim 9 wherein the gas mixture is selected from the group consisting of blends of oxygen with nitrogen and/or argon and/or helium.

11. The process of claim 9 wherein the zirconium-based alloys may contain varying amounts, preferably from 0.01 to 3% by weight of the total weight of the alloy, of alloying elements such as tin, niobium, chromium, iron, oxygen, and nickel.

12. The process of claim 9 wherein the metal halides produced in the oxidation and neutralization reactions are different and they are regenerated independently.

13. A process to convert alkanes to primary alcohols of the same carbon number wherein there are halogenation (reaction of halogen with the alkanes), oxidation (reaction of alkyl halide with a metal oxide), and regeneration (reaction of metal halide with air, oxygen, or other oxygen gas containing mixtures) reactions which take place in the process and that these reactions, at least, are carried out in reactors made from metallurgy including zirconium and/or zirconium-based alloys that contain varying amounts of alloying elements such as tin, niobium, chromium, iron, oxygen, and nickel.

14. The process of claim 13 wherein the zirconium-based alloys may contain varying amounts, preferably from 0.01 to 3% by weight of the total weight of the alloy, of alloying elements such as tin, niobium, chromium, iron, oxygen, and nickel.

15. A process for the production of primary alcohols from alkanes which comprises the steps of:
a) halogenating a linear alkane, branched alkane, or mixture of linear and branched alkanes to produce a mixture of primary mono-haloalkanes, internal mono-haloalkanes, unreacted alkanes, hydrogen halide, and possibly multi-haloalkanes;
b) separating the primary mono-haloalkanes from the mixture of step a) by distillation or other appropriate separation step(s);
c) oxidizing the separated primary mono-haloalkane with a metal oxide or combination of metal oxides and water (and possible hydrogen halide) to convert the aforesaid primary mono-haloalkane to a mixture of products that contains primary alcohols, unconverted primary mono-haloalkanes, and possibly other reaction products, and a partially halogenated metal oxide and/or metal halide or a mixture of partially halogenated metal oxides and/or metal halides;
d) separating and regenerating the partially halogenated metal oxide and/or metal halide or a mixture of partially halogenated metal oxides and/or metal halides to a metal oxide or mixture of metal oxides and molecular halogen by reaction with air, oxygen or gas mixtures containing oxygen gas, wherein the halogen produced and/or the metal oxide may be recycled; and
e) removing the unreacted primary mono-haloalkane from the reaction mixture and then purifying the primary alcohol;
wherein steps a), c) and d) are carried out in equipment which is made from metallurgy which includes zirconium and/or zirconium based alloys.

16. The process of claim 15 wherein the zirconium-based alloys may contain varying amounts, preferably from 0.01 to 3% by weight of the total weight of the alloy, of alloying elements such as tin, niobium, chromium, iron, oxygen, and nickel.

17. The process of claim 15 wherein the gas mixture is selected from the group consisting of blends of oxygen with nitrogen and/or argon and/or helium.
